(19) United States
(12) Patent Application Publication
(10) Pub. No.: US 2011/0137135 A1
(43) Pub. Date: Jun. 9, 2011

(54) Context Aware Physiological Monitoring

(76) Inventor: Michael J. McGrath, Dublin (IE)

(21) Appl. No.: 12/633,933

(22) Filed: Dec. 9, 2009

(51) Int. Cl. A61B 5/11; A61B 5/01; A61B 5/024; A61B 5/022

(52) U.S. Cl. 600/301; 600/595; 600/549; 600/508; 600/490

(57) ABSTRACT

To make home patient care more successful, a machine may enable context aware physiological monitoring at home. For example, in one embodiment, the machine only allows the physiological monitor measurements to be made when the patient has proven that the patient has not undergone strenuous activity in a period preceding the measurement. That activity may skew the measurement and make the measurement unreliable. Without the machine information, the clinician may be unaware of the physical activity because the patient is not present in his or her office and could misdiagnose or misprescribe based on incorrect information.

### BACKGROUND

This relates to physiological monitoring.

Physiological monitoring is the monitoring of a patient's physiological condition for purposes of medical diagnosis and/or treatment. Typically, a patient's blood pressure, pulse rate, pulse oximetry, and peak flow may be monitored. These measurements may be important to correct medical diagnosis and treatment.

However, physiological monitoring by the patient, at home, is sometimes prone to errors. Some of these errors may be due to improper operation of the equipment and to improper procedures in association with that equipment.

Because of the high cost of hospitalization, it is desirable to enable patients to stay at home and to treat them remotely, if possible. However, the ability to do so may be compromised where the physiological measurements are not sufficiently accurate.

### BRIEF DESCRIPTION OF THE DRAWINGS

FIG. 1 is a schematic depiction of one embodiment of the present invention;

FIG. 2 is a schematic depiction of the wearable kinematic sensor in accordance with one embodiment; and

FIG. 3 is a flow chart for one embodiment.

### DETAILED DESCRIPTION

Some home-based physiological measurements, including blood pressure, pulse oximetry, and peak flow, may be recorded in a home environment. These measurements may then be transferred electronically to a clinician to monitor the patient's progress and to assess treatment options. In some cases, the context in which these measurements were taken is important in interpreting the readings.

The clinician normally has no knowledge about what the patient was doing prior to taking the measurements, in the case where the measurements were transmitted by the patient from the patient's home over an electronic transport medium. If the patient walked upstairs, worked out, or did other activities prior to taking the physiological measurements, their accuracy may be questioned. The type of activity and the magnitude of the activity can have a significant impact on the physiological monitor's reading. Specifically, physical activity typically raises blood pressure.

This missing contextual information can be provided by a body kinematic sensor that captures and logs physical activity. Then, when the physiological monitor data is transferred to the clinician, it may be accompanied with the kinematic information that enables the clinician to determine what the patient was doing at times proximate to the measurements in some embodiments.

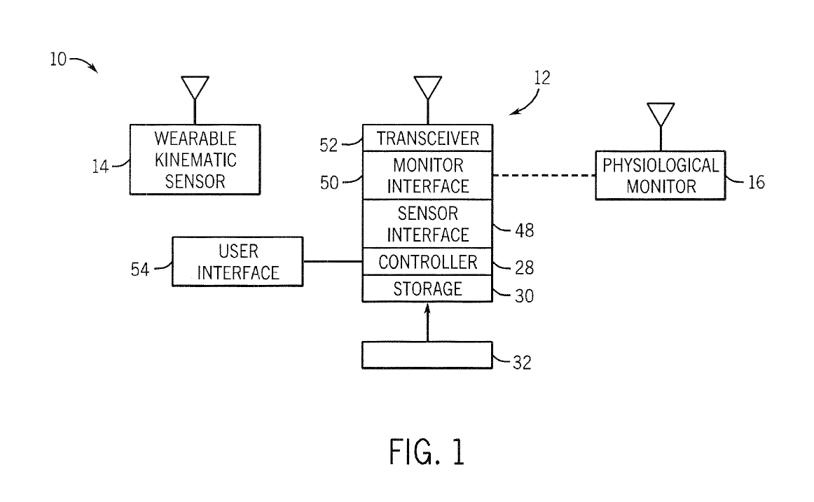

Thus, referring to FIG. 1, a home physiological monitoring system may include a wireless center 12 in one embodiment. In some cases, a wired center may be used as well. However, a wireless center may communicate wirelessly with physiological monitor 16. Examples of physiological monitors include blood pressure cuffs, pulse oximetry devices, and peak flow measurement devices.

The measurements taken by these devices may be transferred wirelessly or by a wired connection to the wireless center 12. The wireless center 12 may also be in wireless communication with a wearable kinematic sensor 14. The wireless communication may be short range wireless communication (such as a Bluetooth connection) or long range wireless communication (such as a cell phone connection), as desired.

The wireless center 12 may include a processor or a control 28 and a storage 30. The storage 30 may store a sequence of instructions 32 in some embodiments. In such case, the storage 30 may comprise a computer readable medium storing instructions for execution by the control 28. A user interface 50 may be a display, a speaker, a microphone, a keyboard, or a mouse, to mention a few examples. A monitor interface 50 may process wired or wireless signals from the physiological monitors 16. The sensor interface 48 processes signals from the sensor 14.

The wireless center 12 may be in communication over wired or wireless connections with a clinician. Thus, a patient may monitor his or her physiological conditions at home, enable these to be transferred to the wireless center, and then the wireless center 12 may transfer them over a suitable medium to the clinician.

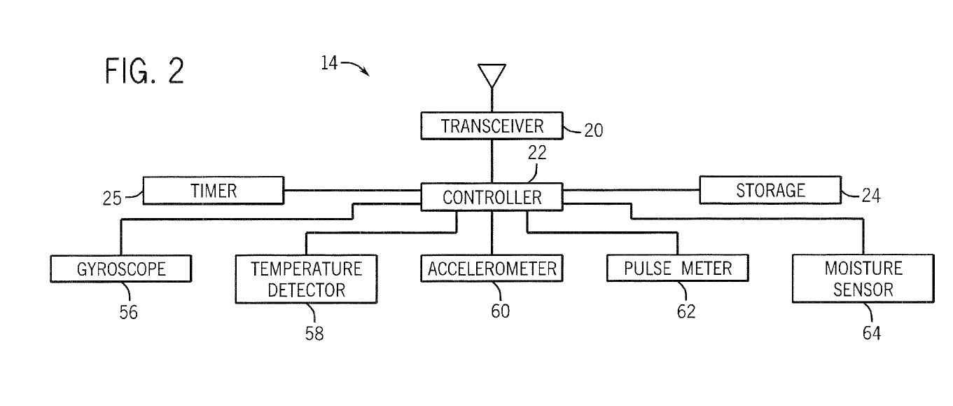

Referring to FIG. 2, the kinematic sensor 14 may include an accelerometer 26, a temperature sensor 58, a pulse meter 62, a gyroscope 56, and a moisture sensor 64, coupled to a controller or processor 22. The moisture sensor 64 may, for example, sense ambient air moisture for use in connection with peak flow readings. A storage 24 may enable storage, over a period of time, of the activity readings. The readings may be stored in association with a time from the timer 25.

Thus, a log may be made of what activities were done at what time in one embodiment. This log may then be combined with a log indicating the time when the physiological measurements were taken. The physiological monitoring log may be prepared by a timer equipped monitor 16, or by the wireless center 12 logging the time when the measurement is received. It may be determined whether physical activity was undertaken in proximity to the physiological measurements.

In one embodiment, the wearable kinematic sensor is a Shimmer wireless sensor platform from Shimmer Research, Dublin, Ireland. The Shimmer wireless sensor platform includes a low power microprocessor that controls device operation and facilitates communication with peripheral devices. It captures sensor data from analog-to-digital converter channels. It then periodically conveys this data over a wireless link to the wireless center 12. In some cases, the device is relatively small and light and can be carried on the person's body or clothing. It includes a three axis microelectromechanical accelerometer for sensing movement.

In one embodiment, the sensor 14 communicates over a Bluetooth wireless connection with the wireless center 12. The wireless center processes the information it receives and coordinates the log of time and activity with the time when physiological monitor readings were received. The wireless center 12 then can combine all of this information and transmit it in a useful form to the clinician in some embodiments.

In one embodiment, if no activity levels above a threshold were recorded over a predetermined measurement window, such as fifteen minutes prior to the physiological measurement; the physiological monitor's measurement can be transmitted to clinician. Otherwise, the patient may be asked to sit restfully for a period of time prior to taking the measurements again.

The sensor 14 may provide patient activity information upon request over a preset time period, such as fifteen minutes. The information may be generated by an accelerometer 26 that, in one embodiment, may be a three axis accelerometer. In one embodiment, the activity information may be refined by determining the minimum and maximum accelerometer values over the predetermined time window. The average of those values may be determined and compared to a predetermined limit. These analyses can be done in the sensor 14 or the wireless center 12.

The limits may be determined by looking at average values for various activities such as walking, running, climbing stairs, etc. Thus, in some cases, the readings can be used to determine the activity type. The data may be better characterized, in some embodiments, using data from the gyroscope 56.

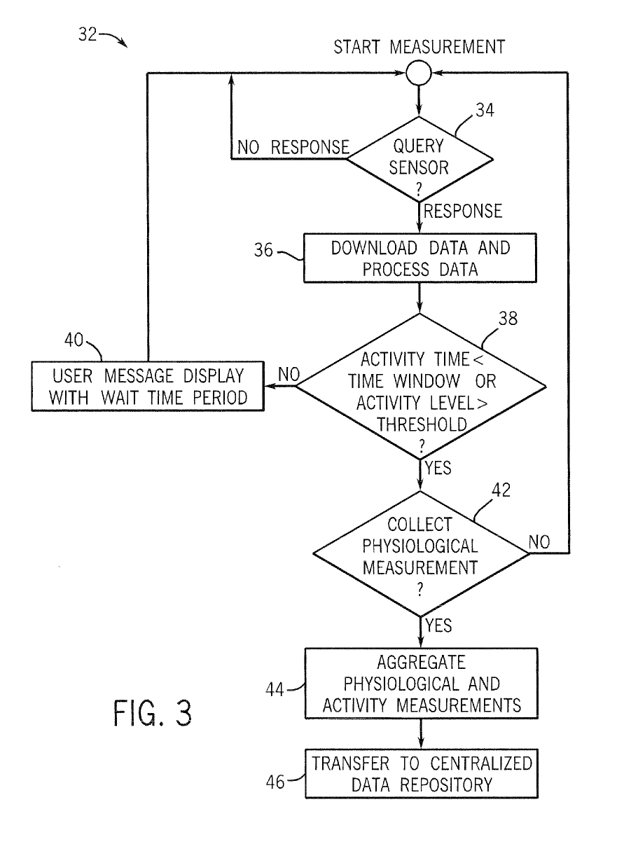

Referring to FIG. 3, a sequence, in accordance with one embodiment, is depicted. The sequence, in one embodiment, may be implemented by instructions stored on a computer readable medium such as the storage 30. In other cases, the sequence may be implemented in hardware.

Initially, at diamond 34, the wearable kinematic sensor 34 is queried to determine whether or not there was a response to a ping for activity data, or not. This query may be in response to a user request to take a physiological monitor reading, in one embodiment. In some cases, the sensor may only provide information at certain intervals in order to save power. In other cases, the sensor only provides data upon request.

If a response was received from the sensor 14, the data may be downloaded and processed, as indicated at block 36. Thus, a log of time and activity may be received for a preceding time period of interest, which may be fifteen minutes in one embodiment. That information may be processed to determine an average activity level and to compare that average activity level to a preset threshold level, in one embodiment.

Thus, when queried, the sensor only sends the relevant data, i.e. the activity measurement for the preceding pertinent window, e.g. fifteen minutes in one embodiment. In such case, the wireless center 12 may only pull the data from the sensor 14 when it has received a request to use a physiological monitor reading. In that case, the sensor may be programmed only to provide activity information for a predetermined time, such as fifteen minutes of data, in response to a request from the wireless center 12.

The activity time or time window of activity level greater than the threshold is then checked in diamond 38. If there was activity above a threshold within the time window when activity should be limited, a user message may be displayed with a wait time period, as indicated at block 40. In such case, the user is asked to wait a preset time (e.g. fifteen minutes) before seeking to take the physiological measurements.

If there was no activity in excess of the threshold within the activity time or time window, the user may be advised through the user interface to proceed to take the physiological monitor measurement. The physiological measurements may be collected from the physiological monitors, as determined in diamond 42. If the information is successfully collected, it can be aggregated with the activity measurements if desired and transferred to a centralized data repository in blocks 44 and 46.

In some embodiments, three separate units may be used, including the wireless center, the body wearable sensor, and the physiological monitor or monitors. However, in other embodiments, less than three discrete units may be provided. For example, the physiological monitor may include the wireless center. In other embodiments, a wearable device may perform all of the functions of the physiological monitor, the wireless center, and the kinematic measurement. In other embodiments, more than three units may be used.

In other embodiments, additional information may be collected by the wearable sensor 14. For example, in addition to kinematic measurements, a wearable sensor may also provide information about body temperature, moisture or sweating, ambient moisture, pulse, etc, which may be useful in analyzing the physiological monitoring measurements or for other purposes.

References throughout this specification to “one embodiment” or “an embodiment” mean that a particular feature, structure, or characteristic described in connection with the embodiment is included in at least one implementation encompassed within the present invention. Thus, appearances of the phrase “one embodiment” or “in an embodiment” are not necessarily referring to the same embodiment. Furthermore, the particular features, structures, or characteristics may be instituted in other suitable forms other than the particular embodiment illustrated and all such forms may be encompassed within the claims of the present application.

While the present invention has been described with respect to a limited number of embodiments, those skilled in the art will appreciate numerous modifications and variations therefrom. It is intended that the appended claims cover all such modifications and variations as fall within the true spirit and scope of this present invention.

What is claimed is:

1. An apparatus comprising:
a monitor interface to receive a physiological monitor measurement;
a transmitter to transmit the measurement to a remote location; and
a sensor interface to obtain information about kinematic activity from an activity sensor.

2. The apparatus of claim 1 including an activity sensor having an accelerometer to detect user motion, said activity sensor being mountable on the human body.

3. The apparatus of claim 2 wherein said activity sensor includes a gyroscope.

4. The apparatus of claim 2 wherein said activity sensor includes at least one of a temperature sensor, a moisture sensor, and a pulse meter.

5. The apparatus of claim 1 wherein said monitor interface to request kinematic activity information from the activity sensor in response to a request to provide a physiological monitor measurement.

6. The apparatus of claim 1, said sensor interface to receive information about user activity for a preset amount of time prior to the request for that information.

7. The apparatus of claim 1 wherein said sensor interface to determine the average activity level and to compare said average activity level to a threshold to determine whether excessive activity has occurred.

8. The apparatus of claim 1 including a controller to determine whether excessive activity has occurred and, if so, to provide an indication that the physiological monitor measurement should be delayed.

9. The apparatus of claim 1 to combine physiological monitor measurements and activity sensor information and to automatically transmit the combined data to a remote location.

10. The apparatus of claim 1 wherein said physiological monitor measurements include a pulse oximeter, a blood pressure cuff, and a peak flow meter.

11. A computer readable medium storing instructions executed by a computer to:
in response to a request to provide a physiological monitor measurement, request information about user activity; and
if said user activity is in excess of a threshold, providing an indication to delay the physiological monitor measurement.

12. The medium of claim 11 further storing instructions to combine the physiological monitor measurement and the activity information and to transmit the information to a remote location.

13. The medium of claim 11 further storing instructions to determine an average activity level, to compare that said average activity level to a threshold, and to determine whether excessive activity has occurred.

14. The medium of claim 11 further storing instructions to control the taking of physiological monitor measurements until an activity level is below a threshold.

15. A method comprising:
receiving a request for a physiological monitor measurement;
in response, obtaining information about the user's activity level from a body wearable sensor; and
determining whether the physiological monitor measurement should be taken based on the activity level information.

16. The method of claim 15 including providing an indication that the physiological monitor measurement should not be taken for a period of time.

17. The method of claim 15 including receiving a physiological monitor measurement, combining it with activity level information and transmitting the combined information to a remote location.

18. The method of claim 15 including determining an average activity level and comparing the average activity level to a threshold.

19. The method of claim 15 wherein obtaining information about the user's activity level includes sending a wireless message requesting information from the body wearable sensor.

20. The method of claim 15 including obtaining additional information from the body wearable sensor including at least one of a temperature measurement, a pulse measurement, and ambient moisture measurement, or a gyroscope measurement.
